SEQUENCE LISTING (10 sequences)

SEQ ID NO: 1 - DNA, 23 bp, Artificial Sequence
  fimW-F primer
aacagtcactttgagcatgggtt

SEQ ID NO: 2 - DNA, 22 bp, Artificial Sequence
  fimW-R primer
gagtgactttgtctgctcttca

SEQ ID NO: 3 - DNA, 24 bp, Artificial Sequence
  UP primer
atgaaacgttcacttattgctgct

SEQ ID NO: 4 - DNA, 23 bp, Artificial Sequence
  LO primer
ttaattataagataccaccatta

SEQ ID NO: 5 - DNA, 35 bp, Artificial Sequence
  FanBamUP (PBR) primer
cacggatcctggagaatctagatgaaaaaaacact

SEQ ID NO: 6 - DNA, 37 bp, Artificial Sequence
  FanSalLO (PBR) primer
cgcgtcgactcatataaatgttacagtcacaggaagt

SEQ ID NO: 7 - DNA, 22 bp, Artificial Sequence
  F primer
gctagcatgaaaaaagcattgt

SEQ ID NO: 8 - DNA, 27 bp, Artificial Sequence
  R primer
ggatcctcagaaatacaccaccacccg

SEQ ID NO: 9 - DNA, 19 bp, Artificial Sequence
  FimA-H UP1 primer
atgaaaattaaaactctgg

SEQ ID NO: 10 - DNA, 21 bp, Artificial Sequence
  FimA-H LO1 primer
ttattgataaacaaaagtcac

(19) United States
(12) Patent Application Publication
(10) Pub. No.: US 2023/0193194 A1
(43) Pub. Date: Jun. 22, 2023

(54) GENERIC INERT BIO-VECTOR SALMONELLA SP. AND POTENTIAL USES THEREOF

(71) Applicant: YANGZHOU UNIVERSITY, Jiangsu (CN)

(72) Inventors: Guoqiang ZHU, Jiangsu (CN); Bin YANG, Jiangsu (CN); Yang YANG, Jiangsu (CN); Xia MENG, Jiangsu (CN); Pengpeng XIA, Jiangsu (CN); Qiangde DUAN, Jiangsu (CN); Xiaofang ZHU, Jiangsu (CN)

(73) Assignee: YANGZHOU UNIVERSITY, Jiangsu (CN)

(21) Appl. No.: 17/642,238

(22) PCT Filed: Dec. 28, 2020
(86) PCT No.: PCT/CN2020/140033

§ 371 Date: Mar. 11, 2022

(30) Foreign Application Priority Data
May 19, 2020 (CN) 202010427735.8

(51) Int. Cl. C12N 1/20; G01N 33/569

(52) U.S. Cl. CPC C12N 1/205; G01N 33/56916; C12R 2001/42

(57) ABSTRACT

A generic inert bio-vector Salmonella sp. S9H and potential uses thereof are provided. The generic inert bio-vector Salmonella sp. S9H is derived from a continuous in-vitro culture of an inert bio-vector bacterium Salmonella sp. S9 by using LB solid and liquid culture media for passage to the fortieth generation. With a quantity of bacteria at a working concentration, the S9H does not cause non-specific agglutination reactions in sera or whole blood derived from humans, mice, cattle, pigs and poultry (including chickens, ducks, geese, turkeys, pigeons and quails); moreover, S9H has a property of carrying, and expressing and displaying different antigen factors derived from humans, mice, cattle, pigs and poultry (including chickens, ducks, geese, turkeys, pigeons and quails) on the surface thereof .

### TECHNICAL FIELD

The present invention belongs to the fields of biomedicines and detection technology, and particularly relates to a bio-generic inert vector Salmonella sp. and potential uses thereof. The generic inert bio-vector Salmonella sp. does not cause non-specific agglutination reactions in sera or whole blood derived from humans under different genetic backgrounds and multiple animals, such as, human, mice, cattle, pigs and poultry (including chickens, ducks, geese, turkeys, pigeons and quails) with a quantity of bacteria at a working concentration.

### DESCRIPTION OF RELATED ART

In the studies on epidemic disease prevention and control and epidemiology, the serological detection technology is always used to diagnose whether an animal is infected with or carries a specific pathogen. Agglutination test is a kind of classical and rapid diagnosis method by serology which has been widely used in medicine and veterinary clinical diagnostics. Agglutination test has the following principle: bacterial particulate antigens bind to the corresponding serum antibodies in the presence of electrolytes and at appropriate temperature to occur agglutination and coagulation phenomena within several minutes, thus forming agglutinated pieces or particles; and the reaction result can be observed and determined by naked eyes only. The antigen participating in the reaction is called an agglutinogen, and the antibody is called an agglutinin. Plate agglutination test is a kind of qualitative method which is widely used in agglutination reaction more. A drop of diagnosed serum (containing a given antibody) and a bacterial suspension to be detected is respectively dripped on a clean transparent glass plate, and slightly mixed well with the same amount (volume), staying for 2 minutes at room temperature; if there is a particulate agglutination visible to the naked eye, it is positive agglutination reaction. The plate agglutination test is usually used for bacterial identification and antigen typing. On the contrary, a given diagnosed antigen can be also used to detect the presence of the corresponding antibody in the serum or whole blood to be detected. The glass plate agglutination reaction for the diagnosis of infection of Bacterium burgeri and Salmonella pullorum/Salmonella gallinarum whole blood plate agglutination test, and the like are usually used in medicine and veterinary clinical diagnostics.

The whole blood plate agglutination test always serves as a spot rapid test method. With simple operation, a drop of whole blood is collected on the spot and added with a drop of agglutinogen, and then the glass plate is shaken slightly; moreover, the reaction result can be observed and determined within two minutes. With low cost, the production cost for the direct detection of a single sample is 0.1 Yuan (Chinese dollar) around; field monitoring and detection can be completed without any extra test equipment, let alone expensive laboratory instrument and equipment. With the above advantages, the whole blood plate agglutination test is always extensively applied in the monitoring and detection of vertical transmission diseases in breeding poultry production. For example, in the detection and eradication of Salmonella pullorum infection, the whole blood plate agglutination test always serves as a representative and classic agglutination test for the rapid screening of chicken infected with Salmonella pullorum (antibodies) in large-scale chicken flocks. Because of its convenience and practicality in detection inside a chicken coop and beside an enclosure, the whole blood plate agglutination test has incomparable clinical application advantages. Moreover, the whole blood plate agglutination test played an important role in the Pullorum Disease Eradication of National Poultry Improvement Plan of the United States. But it should be noted that full-bacterial antigens have the drawback of multiple components complexity and the technique for target detection of antibodies by O antigen has poor sensibility. In fact, the agglutinogen detection has certain limitation in practical application. It has reported that there are many kinds of non-specific cross reactions in antigen diagnosis by agglutination; and the detection result of each batch is unstable and has poor repetition results; the detection result is influenced by multiple factors, such as, difficultly judged weakly positive results and leak detection caused by poor sensitivity. Meanwhile, in consideration of the O antigen oligosaccharide, poor antigenicity; and in the three components of O antigen, namely, O₁, O₉ and O₁₂, O₁₂ have three variants including a standard type, a variant type and an intermediate type in the Salmonella pullorum, leading to weak specific matching recognizing reaction between the diagnosed antigen strain and the infected serum. Especially, it should be noted that O antigen of bacteria has limited spatial conformation, limited antigen displaying, and presence of O inagglutinability because of interference of the inherent O non-agglutination factor from the surface of the O antigen such as fimbriae, capsule, membrane proteins. Thereby, the agglutination diagnostic has low sensitivity, which is only relatively sensitive to the detection effect of the infected adult chicken flocks, and the detection and eradication need to be performed in the egg laying process of breeding hens rather than young chicken flocks, while for the detection of infection-induced antibodies of chicks, there probably exists higher leak detection and detection errors, and inconsistent detection results of each batch due to limited sensitivity.

In preliminary study, the applicant has used the commercialized Salmonella pullorum/Salmonella gallinarum applied clinically most widely to make stained agglutinogens for plate agglutination test, thus detecting a same batch of 200 sera from a certain chicken house in twice at different time. It has been found that the total consistent rate of the two batches of the detection results is only 81%, prompting that the two detection results are unstable and the consistency is unsatisfactory. Compared with the detection results of the ELISA kit from Netherlands’ BioChek for Salmonella sp. D sero-group, it is found that the total consistent rate of the detection result is only 79.5%, the positive consistent rate (relevance ratio or sensibility) is 75.2-79.4%, and the negative consistent rate is 79.5-85.5%. The above detection result and comparative analysis indicate that the sensitivity, specificity, repetition stability and result accuracy are not up to a relatively ideal level when the commercialized agglutinogen is used to detect the serum antibody infected with Salmonella pullorum/ Salmonella gallinarum. The above results hint that the detection result of the existing commercialized agglutinogen has a certain degree of or sometimes more obvious false-positive result caused by non-specific reaction detection and false-negative result caused by leak detection. That is, the degree of accuracy of the detection result of the agglutinogen is to be further improved. The primary cause is that agglutinogens applied in the agglutination test currently are all full-bacterial antigens, and are the bacterial particulate antigens compounding multiple different components instead of a single O1, O9, or O12 antigen. Theoretically speaking, such kind of multi-component full-bacterial antigen have homologous and same components with the same family & genus, and other family & genus of species (especially in Enterobacteriaceae), which will cause non-specific cross reactions to a certain extent. Moreover, it is worth noting that since the agglutinogen is required to contain a higher concentration of bacterial quantity at a working concentration and thus, causes non-specific cross reaction, the disadvantages of the non-specific cross reaction will inevitably influence and even significantly interference with the detection and diagnosis results, thereby seriously affecting the eradication effect of epidemic diseases and the implementation of the eradication process of epidemic diseases.

In preliminary study, the inert bio-vector bacterium Salmonella sp. S9 researched by the applicant has a non-agglutination effect on chicken sera under different genetic backgrounds only within a certain range of concentration, but may have different degrees of agglutination to other animals. Therefore, the inert bio-vector bacterium Salmonella sp. S9 is only used the development of a chicken agglutination experiment and uses thereof; and the use is limited to some extent.

To sum up, it is very urgent and necessary to research and develop a detection system to replace the existing classic agglutination test based on the non-specific cross reaction of the Salmonella pullorum full-bacterial antigen agglutination test and the limited sensitivity of the targeted antibody detection by O antigen of bacteria, thus improving the specific and sensitive accuracy. Further, the premise is to research and develop a generic inert bio-vector bacterium which does not cause non-specific agglutination reactions in sera or whole blood derived from humans and various sources of animals. Further, the generic inert bio-vector bacterium can carry, and express and display a single antigen factor and specifically target different pathogenic bacterial infections (antibodies) on the surface thereof, i.e., Salmonella pullorum infection (antibody). Such kind of recombinant generic inert bio-vector bacterium is used to replace the Salmonella pullorum full-bacterial antigen as an agglutinogen, which can precisely and specifically improve the specificity and sensitivity of the agglutinogen reaction in the premise of retaining the advantages, such as, visual and rapid agglutination reaction results, simple operation and on-site test. The agglutination test with such kind of generic inert bio-vector bacterium as a vector can perfect the monitoring, detection and eradication of Salmonella pullorum/Salmonella gallinarum. Such kind of generic inert bio-vector bacterium as a vector can be used to develop specifically-targeting different pathogenic bacterial infections (antibodies). Such novel monitoring and detection method for agglutinogen test have great potential application prospect in diagnosis and detection of human and lots of animal diseases.

### SUMMARY

The objective of the present invention is as follows: it is very urgent and necessary to improve and perfect the specificity, sensibility, repetition stability and detection result accuracy of the agglutination test widely used in the fields of human and animal disease diagnosis and detection. Therefore, the inventor obtains a Salmonella sp. S9H with the features of a generic inert bio-vector by the alternative culture of an inert bio-vector Salmonella sp. S9 for passage to 40 generations by using LB agar and liquid media. The generic inert bio-vector bacterium is featured as follows: with a quantity of bacteria at a working concentration, the generic inert bio-vector bacterium does not cause non-specific agglutination reactions in sera or whole blood derived from human and multiple animals including mice, cattle, pigs and poultry, and the generic inert bio-vector bacterium can express, display and carry a specific antigen factor on the surface thereof, thus targeting a specifically infected antibody. Therefore, the present invention can serve as a generic inert bio-vector bacterium to be applied for the development of an indirect agglutination test for the rapid field monitoring and detection of human and multiple animal infectious antibodies. Thereby, the present invention has wide application aspect. The generic inert bio-vector Salmonella sp. S9H differs from the S9 bacterium in non-agglutination effect with multiple different animals and thus, is called a generic inert bio-vector with a broader range of application.

Technical solution: to solve the above problem, the present invention provides a generic inert bio-vector Salmonella sp.; the generic bio-inert vector Salmonella sp. is derived from a continuous in-vitro culture of an inert bio-vector bacterium Salmonella sp. S9 by using LB liquid and solid culture media for passage to the fortieth generation and above; and the obtained strain is called a generic inert bio-vector Salmonella sp. S9H; and the strain has the same features of the generic inert bio-vector from the fortieth generation to the sixty generation.

The present invention further includes a method for obtaining the generic inert bio-vector Salmonella sp., including the following steps: the generic inert bio-vector Salmonella sp. strain is derived from a continuous in-vitro culture of an inert bio-vector bacterium Salmonella sp. S9 by using LB liquid and solid culture media for passage to the fortieth generation and above;

The generic inert bio-vector Salmonella sp. S9H of the present invention may be cultured in an LB or XLD agar culture medium; and the culture method is as follows: a small amount of the preserved bacterial strain is picked and streaked on an LB or XLD agar culture medium at a culture temperature of 37° C., where, the strain is cultured at 37° C. in the LB agar plate to form round off-white bacterial colonies; and the strain is cultured at 37° C. in the XLD agar plate to form round pink bacterial colonies.

Glass plate agglutination test is used to test the above bacterial suspension of generic inert bio-vector Salmonella sp. S9H to find that there is no self-agglutination phenomenon and the S9H bacterium does not cause non-specific agglutination reactions in a plurality of sera or whole blood derived from humans, mice, cattle, pigs and poultry (including chickens, ducks, geese, turkeys, pigeons and quails).

The present disclosure further includes a generic inert bio-vector indirect agglutination test detection system; and the detection system includes the generic inert bio-vector Salmonella sp S9H. and a complex of the recombinant S9H that may display, express and carry a specific antigen factor on the surface thereof.

The specific antigen factor is one or more from a group consisting of a P factor (Peg fimbriae) of poultry Salmonella pullorum, a K88ac antigen factor of swine derived Escherichia coli, a K99 antigen factor of bovine derived Escherichia coli and an I antigen factor (type I fimbriae) of human Salmonella sp..

The present disclosure further includes a method for construction of the generic inert bio-vector indirect agglutination test detection system, including the following steps:
1) obtaining a coding gene for a specific antigen factor;
2) ligation of the coding gene of the specific antigen factor with an expressing plasmid to obtain a recombinant plasmid;
3) transformation of the recombinant plasmid expressing the specific antigen factor into an S9H electrocompetent cell to obtain an identified recombinant strain as the indirect agglutination test detection system for a generic inert bio-vector.

The coding gene for a specific antigen factor in the step 1) is the coding gene for a P factor of poultry Salmonella pullorum, the coding gene for a K88ac antigen factor of swine Escherichia coli, the coding gene for a K99 antigen factor of bovine Escherichia coli or the coding gene for an I antigen factor of human Salmonella sp..

The present disclosure further includes a use of the generic inert bio-vector Salmonella sp. or the detection system in preparation of an inert bio-vector for an indirect agglutination test for detection of an antigen, or in preparation of an inert bio-vector for an indirect agglutination test for detection of an antibody.

The present disclosure further includes a use of the generic inert bio-vector Salmonella sp. or the detection system in preparation of indirect agglutination test reagents or kits for detection of antigens or antibodies.

The present disclosure further includes a use of the generic inert bio-vector Salmonella sp. or the detection system in preparation of reagents or kits for detection of infections associated with pathogens derived from humans, bovine, pigs, mice or poultry.

The present disclosure further includes a detection kit; the detection kit includes the generic inert bio-vector Salmonella sp. or the detection system.

Beneficial effects: in this present invention, the inert bio-vector S9 is alternatively cultured in LB agar and liquid culture media for passage to 40 generation, and continuously subcultured from the 41st generation to the 60th generation; the obtained strain has the features of a generic inert bio-vector and thus, is called a generic inert bio-vector Salmonella sp. S9H. The S9H has the features of generic bio-inert vector bacteria and is manifested that the generic inert bio-vector Salmonella sp. S9H does not cause non-specific agglutination reactions in sera or whole blood derived from humans, mice, cattle, pigs and poultry (including chickens, ducks, geese, turkeys, pigeons and quails). Moreover, S9H can respectively express, display and carry a P factor of poultry Salmonella pullorum, a K88ac antigen factor of swine Escherichia coli, a K99 antigen factor of bovine Escherichia coli or an I antigen factor of human Salmonella sp on the surface thereof. Therefore, the S9H can be used for the development of an indirect agglutination test detection method for simple, convenient and quick detection of antigens or infection-induced antibodies, for overcoming the technical bottlenecks by improving and perfecting the poor specificity and sensitivity of existing agglutination tests for agglutination antigen and antibody detections, showing wide application value and market prospect.

### BRIEF DESCRIPTION OF THE DRAWINGS

FIG. 1 is a diagram showing agglutination test results of the generic inert bio-vector Salmonella sp. S9H and whole blood derived from different sources (both negative and positive controls for the agglutination test are attached); in which, 1 represents human whole blood; 2 represents bovine whole blood; 3 represents murine whole blood; 4 represents swine whole blood; 5 represents poultry (including chickens, ducks, geese, turkeys, pigeons and quails) whole blood.

FIG. 2 is a diagram showing agglutination test results of the generic inert bio-vector Salmonella sp. S9H and red blood cells derived from different sources (both negative and positive controls for the agglutination test are attached); in which, 1 represents human red blood cell; 2 represents bovine red blood cell; 3 represents murine red blood cell; 4 represents swine red blood cell; 5 represents mixed poultry (including chickens, ducks, geese, turkeys, pigeons and quails) red blood cell.

FIG. 3 is a diagram showing agglutination test results of the generic inert bio-vector Salmonella sp. S9H and sera derived from different sources (both negative and positive controls for the agglutination test are attached); in which, 1 represents human serum; 2 represents bovine serum; 3 represents murine serum; 4 represents swine serum; 5 represents mixed poultry (including chickens, ducks, geese, turkeys, pigeons and quails) sera.

FIG. 4 shows an agarose electrophoretogram of a PCR amplified product of a poultry Salmonella pullorum p gene encoding Peg fimbriae, in which, M: Trans 2K Plus II; 1: p-PCR product.

FIG. 5 shows an enzyme digestion identification electrophoretogram of a recombinant plasmid PMD19T-p containing a p gene of PMD19-T simple vector DNA, in which, Ma: Trans 2K Plus II; Mb: Trans 2K Plus; 1-3: 19T-pNheI single restriction enzyme digest. 4-6: 19T-pBamHI single restriction enzyme digest; 8-10: 19T-p double restriction enzymes digests.

FIG. 6 shows an enzyme digestion identification electrophoretogram of a recombinant plasmid p-pBR322 containing a p gene, in which, M: Trans 15K; 1: p-pBR322 recombinant plasmid; 2: NheI single restriction enzyme digest of p-pBR322 recombinant plasmid; 3: pBR322 plasmid free of a p gene; 4: p-PCR result of the recombinant bacterial solution carrying the recombinant plasmid containing a p gene; 5: a p-PCR positive control of the recombinant plasmid containing a p gene.

FIG. 7 shows a negative staining transmission electron microscope (TEM) observation diagram of a bio-vector bacterium S9H and a recombinant bio-vector bacterium S9H-P expressing poultry Salmonella pullorum p gene on the surface thereof.

FIG. 8 shows a negative staining TEM diagram of K99 fimbriae (46,000×). A, B and C respectively represent a prototype Escherichia coli C83907 expressing K99 fimbriae, a recombinant bio-vector bacterium S9H-K99 expressing Escherichia coli K99 fimbriae, and a recombinant bio-vector bacterium S9H-pBR322 (negative control bacterium) not expressing Escherichia coli K99 fimbriae on the surface thereof.

FIG. 9 shows a SDS-PAGE diagram of thermal extraction of K99 fimbriae. Lane M: protein molecular weight Marker; lanes 1-3 respectively represent a prototype Escherichia coli C83907 expressing K99 fimbriae, a recombinant bio-vector bacterium S9-K99 expressing Escherichia coli K99 fimbriae, and a recombinant vector bacterium S9H-pBR322 (negative control bacterium) not expressing Escherichia coli K99 fimbriae on the surface thereof.

FIG. 10 is a Western blot diagram showing that K99 fimbriae are identified by a mouse anti-K99 fimbriae monoclonal antibody. Lane M: protein molecular weight Marker; lanes 1-3 respectively represent Western blot diagrams that the prototype Escherichia coli C83907 expresses K99 fimbriae, the recombinant bio-vector bacterium S9-K99 expresses Escherichia coli K99 fimbriae, and the recombinant bio-vector bacterium S9H-pBR322 (negative control bacterium) does not express Escherichia coli K99 fimbriae on the surface thereof; after being thermally extracted, the fimbriae are subjected to SDS-PAGE electrophoresis; fimbriae proteins are electro-transformed and subjected to the incubation and recognition reaction of the mouse anti-K99 fimbriae monoclonal antibody.

FIG. 11 shows a negative staining TEM diagram of K88ac fimbriae (46,000×). A represents a recombinant bio-vector bacterium S9H- K88ac expressing Escherichia coli K88ac fimbriae; and B represents a prototype Escherichia coli C83902 expressing K88ac fimbriae.

FIG. 12 shows a SDS-PAGE diagram of thermal extraction of K88ac fimbriae and a Western blot diagram showing that K88ac fimbriae is identified by a mouse anti-K88ac fimbriae monoclonal antibody. Lane M: protein Marker; lane 1: SDS-PAGE that Escherichia coli C83902 expresses K88ac fimbriae; lane 2: SDS-PAGE that the recombinant bio-vector bacterium S9H-K88ac expresses Escherichia coli K88ac fimbriae; lane 3: Western blot showing that the mouse anti-K88ac fimbriae monoclonal antibody identifies the prototype Escherichia coli C83902 to express K88acfimbriae.; and lane 4: Western blot showing that the mouse anti-K88ac fimbriae monoclonal antibody identifies the recombinant bio-vector bacterium S9H-K88ac to express K88ac fimbriae.

FIG. 13 shows a restriction identification electrophoretogram of a recombinant plasmid S9H-I containing human Salmonella sp. I gene. M: trans 15K; 1: S9-I recombinant plasmid; 2: BamHI single restriction enzyme digest of the S9H-I recombinant plasmid; 3: NheI single restriction enzyme digest of the S9H-I recombinant plasmid; and 4: BamHI and NheI double restriction enzymes digests of the S9H-I recombinant plasmid.

FIG. 14 shows a negative staining transmission electron microscope (TEM) observation diagram (type: Philips Tecnai 12,46,000×) of a bio-vector bacterium S9H and a recombinant bio-vector bacterium S9H-I expressing human Salmonella sp. I gene on the surface thereof.

### DESCRIPTION OF THE EMBODIMENTS

Before further describing the embodiments of the present invention, it should be understood that the protection scope of the present invention is not limited to the following specific detailed embodiments. Furthermore, it should be understood that terms used herein are illustrative of the specific embodiments, but not construed as limiting the protection scope of the present invention. Unless otherwise specified, all the technical and scientific terms used herein are the same as the meanings generally appreciated by a person skilled in the art. Besides specific methods, equipment and materials used in the examples, any method, equipment and material in the prior art similar to or equivalent to the methods, equipment and materials used in the examples may be further used to achieve the present invention according to the mastery degree of a person skilled in the art to the prior art and the disclosure of the present invention.

The PBS buffer solution related herein is a 0.01 M phosphate buffer solution having a pH value of 7.4.

The inert bio-vector Salmonella sp. S9 adopted herein has been preserved in the China General Microbiological Culture Collection Center (CGMCC) of Beijing China on Mar. 18, 2019 with the accession No. of CGMCC No.17340; classified and named Salmonella sp. with a code of S9. The preservation evidence of the strain is referring to the patent application with the Application Number of 2019104243698. The inert bio-vector Salmonella sp. S9H adopted herein has been preserved in the China General Microbiological Culture Collection Center (CGMCC) of Beijing China on Oct. 19, 2020 with the accession No. of CGMCC No.20915; classified and named Salmonella sp. with a code S9H.

### Example 1 Obtaining and Verification of the Generic Inert Bio-Vector Salmonella sp S9H

An inert bio-vector Salmonella sp. S9 (accession No.: CGMCCNo.17340) was inoculated on an LB liquid culture medium and shaken for 12 h at 37° C., and then 30 µL bacterial solution was sucked and streaked on an LB solid culture medium for culture for 16-18 h at 37° C. to obtain bacterial colonies (passage 2); the passage 2 single colony was picked and inoculated on the LB liquid culture medium, and then subjected to alternative culture for passage to the 40 generations by using LB liquid and solid culture media according to the above same conditions in such a cycle; since the 40th generation, the obtained single colony is a generic inert bio-vector bacterium S9H. In fact, when the bacterium was subcultured to the 60th generation from the 41st generation, any generation of the bacteria had the features of the above Salmonella sp. S9H.

Table 1 Passages of in-vitro culture for the inert bio-vector bacterium S9H and 100 sera derived from different animals and humans

Quantity of negative agglutination reactions

| Passages of the S9 strain | Chicken-derived serum | Duck-derived serum | Bovine serum | Human serum |
|---|---|---|---|---|
| Passage 1 | 78/100 | 75/100 | 63/100 | 51/100 |
| Passage 2 | 78/100 | 75100 | 64/100 | 53/100 |
| Passage 3 | 83/100 | 78/100 | 72/100 | 54/100 |
| Passage 5 | 91/100 | 86/100 | 84/100 | 78/100 |
| Passage 40 | 92/100 | 89/100 | 88/100 | 86/100 |
| Passage 45 | 92/100 | 89/100 | 88/100 | 87/100 |
| Passage 50 | 92/100 | 89/100 | 89/100 | 89/100 |
| Passage 55 | 91/100 | 90/100 | 89/100 | 88/100 |
| Passage 60 | 92/100 | 90/100 | 89/100 | 89/100 |

A fimW gene primer of Salmonella species which has been reported in the literature by the applicant was used to perform PCR amplification identification on the generic inert vector bacterium S9H; 1 mL of the above generic inert bio-vector bacterium S9H colonies were taken and cultured over the night; then the cultured bacterial solution was prepared into a DNA amplification template by a boiling method; fimW fragments were amplified by PCR and identified by 1.5% sugar gel electrophoresis; the size of the target fragment was 477bp. Sequences of the forward and reverse primers synthesized by GENEWIZ in references are as follows:

fimW-F:5′ -AACAGTCACTTTGAGCATGGGTT-3′;

fimW-R:5′ -GAGTGACTTTGTCTGCTCTTCA-3′;

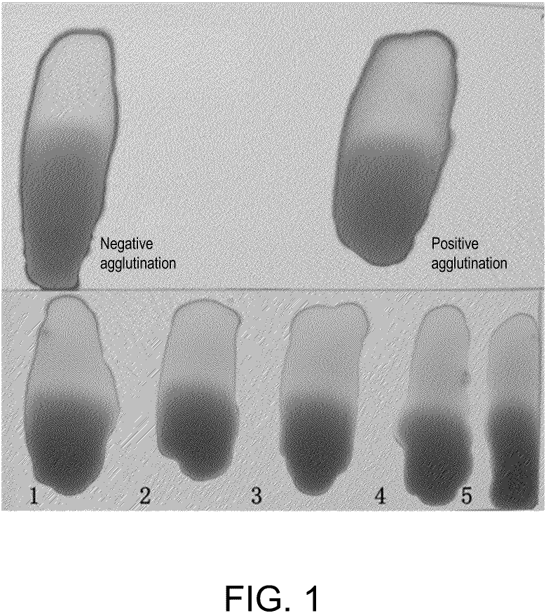

A 20 µL reaction system includes 10 µL 2×Taq Master Mix (Dye Plus) (purchased from Vazyme Biotech Co., Ltd), 1 µL of each fimW-F/R (10 µM), 2 µL DNA template; and 6 µL sterilized ultrapure water as supplementary; PCR reaction parameters: 25 cycles were performed respectively for 5 minutes at 94° C., 30 s at 94° C., 30 s at 55° C., and 30 s at 72° C.; then 10 minutes at 72° C., and stored at 4° C. The identification result of the PCR amplified product by agarose gel electrophoresis shows that the S9H strain may amplify fimW fragment bands (FIG. 1) having a consistent size with the standard strain U20 of Salmonella gallinarum. The bands were verified by DNA sequencing.

A single colony of the S9H strain and the Salmonella gallinarum U20 strain was inoculated on an LB liquid medium for shaking culture over the night at 37° C.; Salmonella sp. diagnosed serum purchased from Tianjin Biochip Co., Ltd. was used for the serotype identification and comparison of the O antigen; and no O1, O9 and O12 Oantigens were detected.

Micro-biochemical tubes purchased from Hangzhou Binhe Microorganism Reagent Co., Ltd. were used for biochemical tests. Micro-biochemical reactions were performed for identification and comparison with sucrose, lactose, glucose, raffinose, maltose, mannitol, indole, mannose, citric acid, dulcitol, ornithine, lysine, potassium cyanide, hydrogen sulfide, urea, ONPG, MR test, V-P test, semi-solid agar, Adonis amurensis and nitrate reduction. Table 2 shows a comparison of biochemical properties between the S9H and the poultry Salmonella gallinarum standard strain U20. Results indicate that the two strains have consistent biochemical test results.

Table 2 Comparison of biochemical properties between the S9H and the Salmonella gallinarum standard strain U20

| Strain | Sucrose | Lactose | Glucose | Raffinose | Mannose | Maltose | Mannitol | Citric acid | Dulcitol | Omithine | Lysine | Potassium cyanide | Hydrogen sulfide | Indole | Urea | ONPG | MR | VP | semi-solid agar | Adonis amurensis | Nitrate reduction |
|---|---|---|---|---|---|---|---|---|---|---|---|---|---|---|---|---|---|---|---|---|---|
| S9 | - | - | + | - | + | + | + | - | - | - | + | - | + | - | - | - | + | - | - | - | + |
| U20 | - | - | + | - | + | + | + | - | - | - | + | - | + | - | - | - | + | - | - | - | + |

Note: “-” denotes negative; “+” denotes positive.

### Example 2 Test on the Zero Non-Specific Agglutination Phenomenon Between the Bio-Vector Bacterium Salmonella Sp. S9H and Sera/Whole Blood Derived From Humans and Animals Under Different Backgrounds

The bio-vector bacterium S9 was alternatively subcultured for 40 passages by using LB agar and LB liquid media according to the method of Example 1 to obtain the generic bio-vector bacterium Salmonella sp. S9H, and the bacterial solution was centrifuged for 10 minutes at 4° C. and 4000 rpm, then supernatant was discarded; the bacterial pellet was resuspended with sterile saline solution, centrifuged and washed for three times, then resuspended to concentration gradients of bacterial quantity at different concentrations. The bacterial solution was mixed well with a vortexer before test, and subjected to agglutination test first with sterile saline solution and SPF chicken serum to ensure that the test bacterial solution was free of self- agglutination and non-specific agglutination phenomenon. Several common glass plates with clean surfaces were taken on a super clean bench (20° C.-25° C.); the bio-vector was centrifuged, resuspended and washed for 3 times with a sterile PBS precooled to 4° C., and diluted to a specified bacterial concentration. A drop of (volume varied from 10 µL to 50 µL) bio-vector bacterium S9H at different concentration gradients was sucked with a micropipettor and vertically dripped on a glass plate surface placed horizontally, and then same amount of sera, red blood cells and whole blood to be detected were rapidly added dropwise The bacterial solution was mixed well with the sera, red blood cells and whole blood with a sterile pipette tip, and coated into a sheet shape having a diameter of 1-2 cm, afterwards, the glass plate was smoothly shaken; the test results must be detected and observed within 2 minutes. The determination standards are as follows: within 2 minutes at room temperature, if the bacterial solution produces a flocculent or granular precipitate visible to the naked eye with the sera to be detected, or produces red coagulation granules with the red blood cells and whole blood to be detected, the reaction result is determined positive, otherwise, it is determined negative. Meanwhile, the S9 bio-vector bacterium was used to prepare a bacterial suspension as a control.

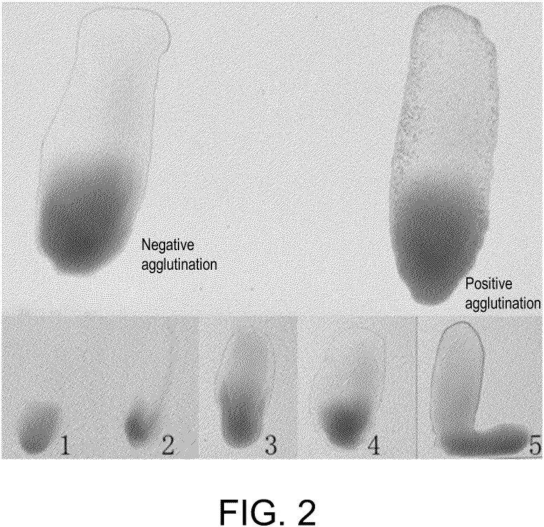

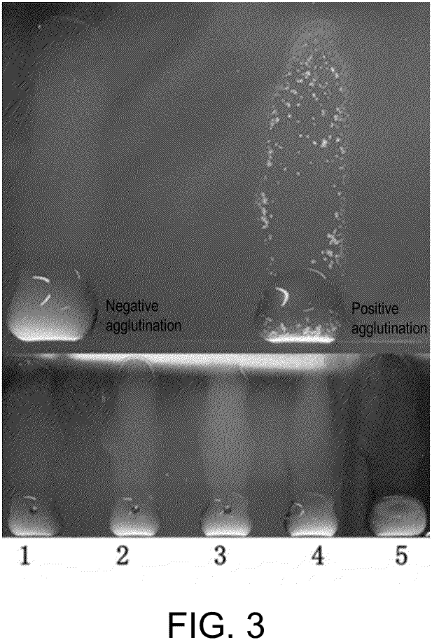

Table 3 shows that the bio-vector bacterium S9 has no self-agglutination phenomenon at different concentrations (500 million CFU/mL to 10 billion CFU/mL); the agglutination reaction results of the bio-vector bacterium S9 are not all negative with the multiple sera, red blood cells and whole blood derived from humans, mice, cattle, pigs and poultry (including chickens, ducks, geese, turkeys, pigeons and quails) under different backgrounds at a concentration of 2.5 billion cfu/mL; but when S9 is up to 5 billion cfu/mL, the bio-vector bacterium S9 has different degrees of agglutination with partial sera and whole blood samples derived from humans and different animals. It is noted that the bio-vector bacterium Salmonella sp. S9H has no self-agglutination phenomenon and has negative agglutination reaction results with multiple sera, red blood cells and whole blood derived from humans, mice, cattle, pigs and poultry (including chickens, ducks, geese, turkeys, pigeons and quails) under different backgrounds at different concentrations (500 million CFU/mL to 10 billion CFU/mL). The above results indicate that the bio-vector bacterium Salmonella sp. S9H does not cause non-specific agglutination reaction with multiple sera, red blood cells and whole blood derived from humans, mice, cattle, pigs and poultry. The above sera, red blood cells and whole blood derived from human and multiple animals under different backgrounds are collected randomly and have negative detection results with the agglutination reaction of the bio-vector bacterium Salmonella sp. S9H; therefore, the Salmonella sp. S9H may be regarded as a generic inert bio-vector Salmonella sp. (FIGS. 1-3).

TABLE 3

Test results of the agglutination reaction between the bacterial suspensions of the bio-vector bacteria S9H and S9 at different concentrations (cfu/mL) and different sources of whole blood and serum

| | | S9H bacterial suspension | | | | | S9 bacterial suspension |
|---|---|---|---|---|---|---|---|
| | | 500 million | 1 billion | 2 billion | 5 billion | 10 billion | 5 billion |
| Human sera and whole | Clinical serum | - | - | - | - | - | +/- |
| | Clinical whole blood | - | - | - | - | - | +/- |
| | A-type blood serum | - | - | - | - | - | +/- |
| | A-type whole blood | - | - | - | - | - | +/- |
| | B-type blood serum | - | - | - | - | - | +/- |
| | B-type whole blood | - | - | - | - | - | +/- |
| | AB-type blood serum | - | - | - | - | - | +/- |
| | AB-type whole blood | - | - | - | - | - | +/- |
| | O-type blood serum | - | - | - | - | - | +/- |
| | O-type whole blood | - | - | - | - | - | +/- |
| Bovine sera and whole blood | Holstein cow serum | - | - | - | - | - | - |
| | Holstein cow whole blood | - | - | - | - | - | - |
| | Jersey cow serum | - | - | - | - | - | - |
| | Jersey cow whole blood | - | - | - | - | - | - |
| | Yellow cattle serum | - | - | - | - | - | - |
| | Yellow cattle whole blood | - | - | - | - | - | - |
| | Beef cattle serum | - | - | - | - | - | - |
| | Beef cattle whole blood | - | - | - | - | - | - |
| Murine sera and whole blood | BALB/C mice serum | - | - | - | - | - | - |
| | BALB/C mice whole blood | - | - | - | - | - | - |
| | C57BC/6J mice serum | - | - | - | - | - | - |
| | C57BC/6J mice whole blood | - | - | - | - | - | - |
| | DBA/2 mice serum | - | - | - | - | - | - |
| | DBA/2J mice whole blood | - | - | - | - | - | - |
| | ICR mice serum | - | - | - | - | - | - |
| | ICR mice whole blood | - | - | - | - | - | - |
| | Wistar rat serum | - | - | - | - | - | - |
| | Wistar rat whole blood | - | - | - | - | - | - |
| Swine | About g of swine serum | - | - | - | - | - | +/- |
| | About g of swine whole blood | - | - | - | - | - | +/- |
| | Landrace serum | - | - | - | - | - | +/- |
| | Landrace whole blood | - | - | - | - | - | +/- |
| | Duroc serum | - | - | - | - | - | +/- |
| | Duroc whole blood | - | - | - | - | - | +/- |
| | Taihu pig serum | - | - | - | - | - | +/- |
| | Taihu pig whole blood | - | - | - | - | - | +/- |
| | Rongchang pig serum | - | - | - | - | - | +/- |
| | Rongchang pig whole blood | - | - | - | - | - | +/- |
| Poultry sera and whole blood | Yellow-feathered broiler serum | - | - | - | - | - | - |
| | Yellow-feathered broiler whole blood | - | - | - | - | - | - |
| | White-feathered broiler serum | - | - | - | - | - | - |
| | White-feathered broiler whole blood | - | - | - | - | - | - |
| | Hy-Line Brown layer serum | - | - | - | - | - | - |
| | Guangxi Fengyuan Chicken serum | - | - | - | - | - | - |
| | Sandeli Jinmaocao Chicken serum | - | - | - | - | - | - |
| | Lihua Xueshancao Chicken serum | - | - | - | - | - | - |
| | Yellow-feathered cock serum | - | - | - | - | - | - |
| | Duck-derived serum | - | - | - | - | - | - |
| | Duck-derived whole blood | - | - | - | - | - | - |
| | Goose-derived serum | - | - | - | - | - | - |
| | Goose-derived whole blood | - | - | - | - | - | - |
| | Turkey-derived serum | - | - | - | - | - | - |
| | Turkey-derived whole blood | - | - | - | - | - | - |
| | Pigeon-derived serum | - | - | - | - | - | - |
| | Pigeon-derived whole blood | - | - | - | - | - | - |
| | Quail-derived serum | - | - | - | - | - | - |
| | Quail-derived whole blood | - | - | - | - | - | - |

Note: “-” denotes negative; “+” denotes positive.

### Example 3 Test and Verification for Surface Expression and Carrying of the Poultry Salmonella Sp. Antigen Factor P by the Bio-Vector Bacterium Salmonella Sp. S9H

### (I) Amplification of the Encoding Gene P Expressing the Antigen Factor P of Salmonella Sp.

The full length fragment of p gene encoding the antigen factor P were respectively searched and aligned according to the full length genome sequences published in NCBI GenBank, namely, the whole genome sequence (NCBI accession number: CP012347.1) of the Salmonella pullorum ATCC 9120 strain, the whole genome sequence (NCBI accession number: LK931482.1) of the Salmonella pullorum S44987_1 strain, the whole genome sequence (NCBI accession number: CP006575.1) of the Salmonella pullorum S06004 strain, the whole genome sequence (NCBI accession number: CP022963.1) of the Salmonella pullorum QJ-2D-Sal strain, the whole genome sequence (NCBI accession number: AM933173.1) of the Salmonella pullorum 287/91 strain, and the whole genome sequence (NCBI accession number: CP019035.1) of the Salmonella pullorum 9184 strain. Olige7 primer software was used to design the primers of the p gene amplified by PCR. The forward and reverse primers are respectively as follows:

UP: 5′ -ATG AAA CGT TCA CTT ATT GCT GCT-3′

LO: 5′ -TTA ATT ATA AGA TAC CAC CAT TA-3′.

NheI and BamHI restriction enzymes cutting sites and protective bases were respectively added on the 5′-terminals of the forward and reverse primers; and a boiling method was used to prepare a reference strain U20 template of Salmonella gallinarum; the p gene PCR amplification system (p-PCR): 10 µL 5X pfu DNA polymerase buffer, 5 µL dNTP, 2 µL forward primer, 2 µL reverse primer, 2 µL template, 2 µL pfu high-fidelity DNA polymerase (2.5 units/uL), 27 µL deionized water (5X pfu DNA polymerase buffer, dNTP and pfu high-fidelity enzyme were purchased from TransGen Biotech. PCR reaction parameters: 30 cycles were performed for 5 minutes at 94° C., 1 minute at 94° C., 1 minute at 52° C., and 1 minute at 72° C.; then 10 minutes at 72° C., and stored at 4° C.

At the end of the above p-PCR reaction, 2.4 µL rTaq DNA polymerase (5U/µL, purchased from TakaraBio) was added to the system, and Poly A tail was added for reaction for 20 minutes at 72° C.

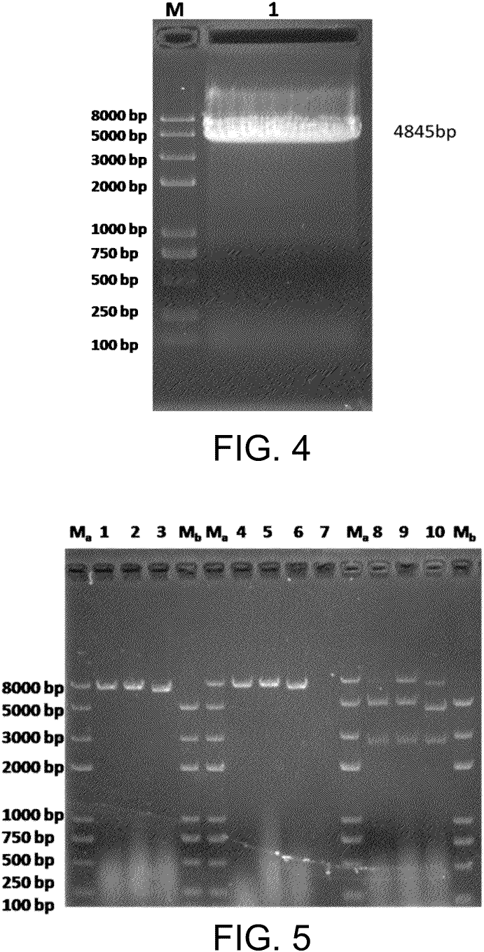

10 µL 6X Loading buffer was added to the above PCR amplified product, and 1% agarose gel electrophoresis was performed at 90 V for 1 h. An UV gel imager was used for observation and target bands were subjected to gel cutting (FIG. 4) according to the operating instruction; the PCR amplified product was recovered by an agarose gel recovery kit; then the recovery product containing PCR amplified DNA gene was preserved for further use at -20° C.

### (II) Construction and Identification of the Recombinant Plasmid 19T-p Containing P Gene

The above obtained PCR amplified product with the addition of A tail was ligated to a PMD19-T simple vector DNA (hereinafter referred to as a 19T vector, and purchased from Promega). 10 µL ligation system is as follows: 1 µL 19T vector, 4 µL recovery product containing PCR amplified DNA gene and 5 µL solution I; the above reaction system was put to a 16° C. metal bath device for ligation over the night.

The ligated product was transferred into DH5α competent cells in the following day by a chemical method; and the operation was as follows: DH5α competent cells at ultralow temperature were placed on an ice for thawing, and 10 µL ligated product was added to the competent cells (the ligated product was added just when the competent cells were thawed), slightly patted to be mixed well, and put on an ice bath for 30 minutes, and subjected to heat stress for 30 s at 42° C., then immediately put on an ice for 2 minutes. The above product was added with 250 µL of an LB solution balanced to at room temperature, incubated for 2 h at 37° C. and 200 rmp, and then centrifuged for 1 minute at 4000 rpm, then supernatant was discarded, and a few of (about 100 µL) supernatant was reserved to resuspend bacterial cells, and coated on an ampicillin LB solid medium, staying over the night at 37° C.

p-PCR identification: whether there was colony growth on the ampicillin LB solid medium and the growth of bacteria were observed; a single colony was picked onto an ampicillin liquid LB for shaking culture for 16 h; 2 µL were taken as a template for the PCR identification of the bacterial solution; the reaction system: 10 µL 2×Taq Master mix (purchased from Vazyme Biotech Co.,Ltd.), 1 µL p gene forward primer, 1 µL p gene reverse primer, 2 µL template (bacterial solution), and 6 µL deionized water. Reaction parameters: 25 cycles were performed for 10 minutes at 95° C., 1 minute at 94° C., and 1 min at 52° C.; then 1 minute at 72° C.; 10 minutes at 72° C. and stored at 4° C. 1% agarose gel electrophoresis was performed for 1 h at 90 V for observation and identification.

Plasmid restriction enzyme digestion and electrophoresis identification: a commercialized kit was used to extract a p gene recombinant plasmid 19T-p; the purified plasmid was subjected to NheI single restriction enzyme digest, NheI and BamHI double restriction enzymes digests (restriction enzymes NheI and BamHI were purchased from TakaraBio), and then identified by agarose gel electrophoresis. NheI single restriction enzymes digest system: 5 µL M buffer, 1 µL NheI, 30 µL plasmid and 14 µL water. Double restriction digests system: 5 µL BglI buffer, 1 µL NheI, 1 µL BamHI, 30 µL plasmid and 13 µL water. 1% agarose gel electrophoresis was performed for 1 h at 90 V for observation and identification after water bath for 3 h at 37° C. (the results are shown in FIG. 5).

### (III) Construction of the Recombinant Plasmid p-pBR322 Containing P Gene

The p-PCR amplified product, positive result of the recombinant plasmid 19T-p containing p gene and the size of the restriction enzyme plasmid in the above step were consistent with the expected values; DNA sequencing was performed for verification; the plasmid pBR322 and the recombinant plasmid 19T-p were subjected to NheI and BamHI double restriction digests, and the restriction digest system was the same as the above (II). After the agarose gel electrophoresis was performed for observation and identification, DNA gel pieces of the target band at 4361 bp and 4845 bp were respectively cut, and DNA of the two target bands was respectively recovered by a commercial kit. DNA T4 ligase reaction system: 1 µL 10X buffer solution, 2 µL enzyme digested pBR322 recovery product, 2 µL p enzyme digested recovery product, 1 µL T4 ligase (purchased from Promega) and 4 µL deionized water. Ligation was performed at 16° C. in a metal bath over the night to obtain a p-pBR322 recombinant plasmid.

### (IV) Construction and Identification of the Inert Bio-Vector Detection System S9H-P Containing p Gene

The ligated product of the above p-pBR322 recombinant plasmid ligated over the night was electro-transformed into competent cells of the bio-vector bacterium S9H; and the detailed operation was as follows:

Preparation of electrocompetent cells S9H: S9H single colony growing on an LB plate over the night was picked, and inoculated onto a 4 mL LB liquid medium for shaking culture for 3 h-5 h at 37° C., then the growth of bacteria was observed. The bacterial solution was inoculated onto a 4 mL LB liquid medium according to 1:100, shaken to OD₆₀₀ at 37° C., 0.4-0.6 h later, put on an ice bath for 30 minutes, and centrifuged for 10 minutes at 4° C. and 4000 rpm, then supernatant was discarded. Precooled 10% glycerin was added for centrifugal washing for three times at 4° C., and 40 µL 10% glycerin was used for resuspending, then the obtained solution was stored temporarily at -70° C. for further use.

Electro-transformation operation: 2 µL p-pBR322 recombinant plasmid was taken and mixed with 40 µL S9H electro-transformed competent cells, then put on an ice bath for 30 minutes; the above mixture was added to 0.1 cm Bio-Rad electric shock cup for electric shock for electro-transformation; then the transformed products were rapidly transferred onto a 1 mL SOC liquid medium, shaken for 4 h at 37° C. and centrifuged for 10 minutes at 4000 rpm; supernatant was discarded; and a few of bottom liquid was reserved and resuspended, then uniformly coated on an ampicillin plate for culture over the night at 37° C.

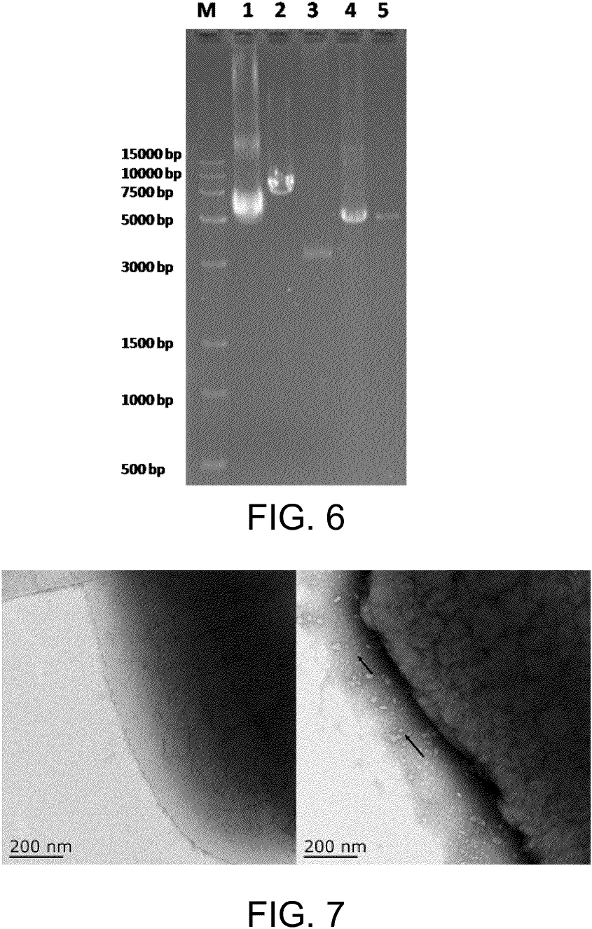

The growth of the bacterial colonies was observed in the following day; the product was amplified by P-PCR and subjected to plasmid DNA enzyme digestion, agarose gel electrophoresis for observation and identification (FIG. 6); after DNA sequencing verification, S9H-P positive single colonies were picked and preserved.

### (V) Identification of the Inert Bio-Vector Detection System S9H-P Expressing a P Factor

The bio-vector bacterium S9H and the S9H-P strain containing p gene inert vector detection system were respectively inoculated onto LB and ampicillin resistant LB agar media, and cultured at 37° C. for 24 h, then a single colony was picked and respectively inoculated onto LB and ampicillin resistant LB liquid media, and placed at 37° C. for shaking culture for 12 h, and subjected to blind passage for 10 generations; a small amount of bacterial solution was sucked to be respectively inoculated onto LB and ampicillin resistant LB liquid media for static culture for 48 h at 37° C., then centrifuged for 2 minutes at 10,000 rpm; precipitates were resuspended with sterile PBS; and a small amount of supernatant was sucked, and suspended on a copper screen, and subjected to negative staining for 5 minutes with phosphotungstic acid. Netherlands Philips Tecnai 12 TEM was used for observation, shooting and result display. P antigen factor component seemed to be not found on the surface of S9H, while an antigenic component (P factor component) appeared and was carried on the surface of S9H-P (FIG. 7).

### Example 4 Test and Verification for Surface Expression and Carrying of the Bovine Escherichia Coli K99 Antigen Factor by the Bio-Vector Bacterium Salmonella Sp. S9H

### (I) PCR Primer Design and Synthesis, Amplification and Cloning of a Fan Operon Gene

Each fragment sequence information of the fan operon of the bovine Escherichia coli K99 fimbriae were searched from the full length genome sequences published in NCBI GenBank, namely, the whole genome sequence (NCBI accession number: CP026929.1) of the Escherichia coli CFS3246 strain, the whole genome sequence (NCBI accession number: NC_017633.1) of the Escherichia coli H10407 strain, the whole genome sequence (NCBI accession number: JPQX01000001.1) of the Escherichia coli 734/3 strain, the whole genome sequence (NCBI accession number: NZ_AGTD00000000.1) of the Escherichia coli UMNF18 strain for alignment and splicing. A pair of primers for the fan operon encoding K99 fimbriae amplified by PCR were designed. The forward and reverse primers respectively contained BamHI and SalI restriction enzymes cutting sites. The primers were synthesized by Shanghai GeneCore BioTechnologies Co., Ltd. Sequences of the forward and reverse primers are respectively as follows:

FanBamUP (PBR): 5′-CAC GGA TCC TGG AGA ATC TAG ATG AAA AAA ACA CT-3′;

FanSalLO (PBR): 5′-CGC GTC GAC TCA TAT AAA TGT TAC AGT CAC AGG AAG T-3′.

Template DNA of Escherichia coli K99 prototype strain C83907 was prepared by a full-bacterial lysis method; then PCR parameters were designed according to a PCR method of amplifying klenow fragment DNA for DNA amplification of klenow fragments. After the PCR amplified product was subjected to 0.8% agarose gel electrophoresis for observation and identification, DNA of the target band was recovered by a kit and ligated to a pMD-18T vector (purchased from Promega), after the DNA was transformed into competent cells DH5α; ampicillin-resistant LB plate was used to screen the positive-assumed resistant clones; DNA sequencing was performed for identification and verification. pMD-18T containing the fan operon gene and the vector plasmid pBR322 were respectively subjected to double restriction enzyme digests with BamHI and SalI; DNA of the two digested products were extracted by chloroform, precipitated by ethyl alcohol, centrifuged and purified, then ligated at 16° C. under the action of a T4 DNA ligase over the night; the ligated product was transformed into the competent cells of the vector bacterium Salmonella sp. S9H; a small amount of recombinant plasmid was extracted from the obtained recombinant bacteria by an alkaline lysis method for identification; and then subjected to single restriction enzyme digest and double restriction enzyme digests, agarose gel electrophoresis for observation and identification, thus identifying whether the constructed recombinant plasmid is correct, and then DNA sequencing was performed for identification and confirmation. The generic inert vector bacterium of the positive recombinant plasmid carrying the fan operon gene was named as S9H-K99. Meanwhile, the pBR322 empty vector was transformed into a bio-vector bacterium S9H to construct a negative control S9H-pBR322.

### (II) Test and Verification for the Agglutination Reaction Mediated by Mouse Anti-K99 Fimbriae Monoclonal Antibody, Surface Expression and Carrying of the Bovine Escherichia Coli K99 Antigen Factor of the Bio-Vector Bacterium Salmonella Sp. S9H

The single colony of the Escherichia coli K99 prototype strain C83907 was picked and inoculated onto a Minimal mineral salt medium; and the single colony of the recombinant bio-vector bacterium S9H-K99 and the single colony of the S9H-pBR322 were picked and cultured on an ampicillin resistant LB liquid medium over the night, and centrifuged at 12,000 rpm; then supernatant was discarded; the obtained product was washed by PBS buffer solution for twice, and resuspended in a proper amount of PBS 5 µL sample was taken and mixed with the sera from different dilution degrees of mouse anti-Escherichia coli K88ac fimbriae monoclonal antibody/polyclonal antibody, F18ab fimbriae polyclonal antibody, F18ac fimbriae polyclonal antibody, and K99 fimbriae monoclonal antibody on the surface of a glass plate, then incubated for 2 minutes at room temperature, and observed under the light to determine the agglutination reaction result. The above monoclonal antibody and polyclonal antibody sera were prepared by the laboratory with reference to the article (Ma Yan, Wang Yiting, Zhao Jing, et al., Preparation of Escherichia coli F4 fimbriae Agglutination Monoclonal Antibodies and Epitope Difference [J]. Journal of Yangzhou University (Volume of Agricultural & Life Sciences), 2017, 38(01): 12-15+34.; Yang Yang, Hou Huayan, Yu Lei, et al., Clone, Expression and Activity of Escherichia coli K99 fimbriae fan Operon [J]. Journal of Microbiology, 2012, 52(12): 1524-1530.) specifically.

The agglutination reaction result indicates that the S9H-K99 recombinant bacterium and the mouse anti-K99 fimbriae monoclonal antibody were subjected to obvious agglutination reaction, but does not cause agglutination reaction with polyclonal antibodies of Escherichia coli K88ac, F18ab, F18ac and Salmonella gallinarum U20 and Salmonella enteritidis C50336 preserved in this laboratory. The above result indicates that the bio-vector bacterium Salmonella sp. S9H expresses and carries the bovine Escherichia coli K99 antigen factor on the surface thereof, while the S9H-pBR322 negative control bacterium does not express the K99 antigen factor on the surface thereof.

### (III) TEM Observation, Test and Verification for Surface Expression and Carrying of The Bovine Escherichia Coli K99 Antigen Factor by the Bio-Vector Bacterium Salmonella Sp. S9H

The Escherichia coli K99 prototype strain C83907, S9H-K99 recombinant bacterium and the S9H-pBR322 negative control bacterium not expressing K99 fimbriae were respectively cultured for 16 h, and then centrifuged to discard the supernatant; then the obtained product was washed for 3 times with PBS buffer solution and then resuspended. Afterwards, a proper amount of bacterial solution was sucked and suspended into a copper grid screen, and subjected to negative staining for 5 minutes with phosphotungstic acid. Philips Tecnail2-twin TEM was used to observe whether the presence and distribution of the fimbriae on the surface of the bacterium.

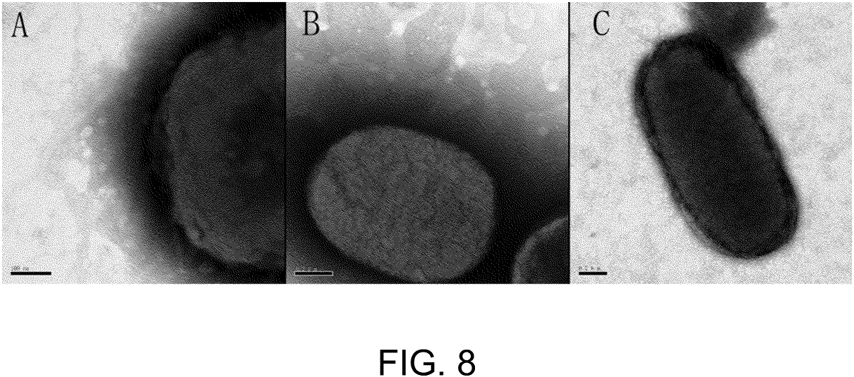

The SEM observation result shows that fimbriae were distributed over the cell surface of recombinant bacterium S9H- K99; the morphology of the fimbriae was more compact than the Escherichia coli K99 prototype strain C83907. The above result indicates that the recombinant bacterium has a high expression quantity of fimbriae on the surface of the recombinant bacterium, while there is no visible fimbriae on the surface of the recombinant bacterium S9H-pBR322 (negative control bacterium) containing the pBR322 plasmid only (FIG. 8).

### (IV) Identification of Fimbriae, SDS-PAGE, Western Blot, Test and Verification for The Surface Expression and Carrying of the Bovine Escherichia Coli K99 Antigen Factor by the Bio-Vector Bacterium Salmonella Sp. S9H Strain

The recombinant bacterium S9H-K99 was treated by a thermal extraction method for 30 minutes at 60° C. to separate and purify the fimbriae protein; 12%SDS-PAGE was performed according to related literatures, and Coomassie Brilliant Blue R250 was used for staining to observe the size of the major structural protein bands expressing the pili. The Escherichia coli K99 prototype strain C83907 served as a positive control; and the recombinant bacterium S9H-pBR322 served as a negative control. The SDS-PAGE result shows that there is a major structural protein band at 18.5KD from the separated and purified recombinant bacterium S9H-K99; and the size of the major structural protein band is consistent with the size of the major structural protein subunit of the K99 fimbriae expressed by fanC, and is also consistent with the size of the major structural protein band of the fimbriae heat-extracted, separated and purified from the Escherichia coli K99 prototype strain C83907; while for the heat-extracted product of the negative control strain S9H-pBR322, there is no corresponding band at 18.5KD after being identified by SDS-PAGE (FIG. 9).

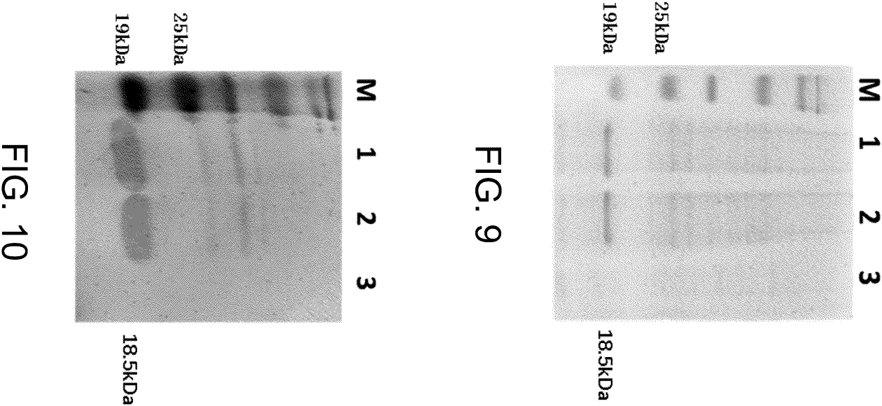

The above thermally extracted, separated and purified fimbriae protein bands were transferred onto a nitrocellulose NC membrane by a BIO-RAD protein band transfer-print system, and then blocked by 10% skimmed milk powder at 4° C. over the night. The NC membrane was washed with PBST for 5 times, and then mouse anti-K99 fimbriae monoclonal antibody diluted by 1:500 as a primary antibody, and goat-anti-mouse IgG-HRP (purchased from SHANGHAI SINO-AMERICAN BIOTECHNOLOGY CO., LTD.) diluted by 1:50 as a secondary antibody were added successively for incubation, then color development was performed with a DAB substrate. Meanwhile, the fimbriae synchronously separated and purified from the Escherichia coli K99 prototype strain C83907 served as a positive control; and the thermally extracted product of the negative control strain S9H-pBR322 served as a negative control. Western blot result indicates that the mouse anti-K99 fimbriae monoclonal antibody may specifically identify the major structural protein bands of fimbriae expressed by the recombinant bacterium S9H-K99 and the Escherichia coli K99 prototype strain, but may not identify the thermally extracted product (FIG. 10) of the negative control strain S9H-pBR322. The above result also shows that the recombinant bacterium S9H-K99 may express and carry the bovine Escherichia coli K99 antigen factor on the surface thereof.

### Example 5 Test and Verification for Surface Expression and Carrying of the Swine Escherichia Coli Antigen Factor K88ac by the Bio-Vector Bacterium Salmonella Sp. S9H

### (I) Design and Synthesis of PCR Amplified Primers

DNAstar software was used to align, analyze and design a pair of PCR primers amplifying the full length of a fae gene operon based on the overall-length genome sequences published in NCBI GenBank, namely, the whole genome sequence (NCBI accession number: CP002729.1) of the Escherichia coli UMNK88 strain, the whole genome sequence (NCBI accession number: EU570252.1) of the Escherichia coli C83549 O149: K88ac strain, and the whole genome sequence (NCBI accession number: CP042627.1) of the Escherichia coli NCYU-25-82 strain, and the sequence information of the fae gene operon encoding swine Escherichia coli K88ac fimbriae published at home and abroad. Forward and reverse primers are respectively as follows:

F:5′ -GCTAGCATGAAAAAAGCATTGT- 3′

R:5′ -GGATCCTCAGAAATACACCACCACCCG- 3′

The forward and reverse primers respectively contained Nhe1 and BamH1 restriction enzymes cutting sites. The primers were synthesized by Shanghai GeneCore BioTechnologies Co., Ltd.

### (II) Preparation for PCR Amplification Template of the Bacterium Chromosome DNA

The DNA of the bacterium chromosome was prepared by a full-bacterial lysis. The reference strain C83902 of Escherichia coli K88ac was shaken for 16-18 h on an LB culture medium, then centrifuged and washed by suspending in ultrapure water, put to an water bath for 10 minutes at 100° C., then placed to an ice bath to be cooled, and centrifuged for 10 minutes at 4° C. and 7000 rpm, then supernatant was taken as a PCR amplification template. The primer has a concentration of 25 pmol/L; the 50 µL reaction system contains 25 µL Buffer, 4 µL dNTP, 1 µL forward primer, 1 µL reverse primer, 5 µL template DNA, and 0.8 µL Long PCR high-fidelity DNA polymerase (5 U/µL, purchased from Vazyme Biotech Co., Ltd); PCR cycle parameters: the template DNA was denaturated for 2 minutes at 94° C., and subjected to 25 cycles in total according to 94° C. (15 s)-50° C. (30 s)-68° C. (3 minutes), and then extended for 20 minutes at 68° C., and stored at 4° C.

### (III) Agarose Gel Electrophoresis, Observation and Identification of the PCR Amplified Product

10 µL PCR amplified product was taken and mixed well with 2 µL 6×loading buffer, and subjected to 0.8% agarose gel electrophoresis (containing 0.5 µg/ml ethidium bromide) with an electrophoresis buffer of 1×TAE, after a constant pressure of 70 V for 1 h, a BIO-RAD gel imager was used to observe and identify the size of the PCR amplified product.

### (IV) Clone Construction of the Positive Recombinant Plasmid pBR322-K88ac Containing the Fae Gene Operon

The PCR amplified product and pBR322 expression plasmid were respectively digested by Nhe1 and BamH1 restriction enzymes, then extracted by phenol/chloroform, precipitated with ethanol and purified; the PCR amplified products after through double restriction enzymes digests were mixed with the pBR322 plasmid according to the amount of 3:1 at the same time; the mixture was ligated by a T4 DNA ligase at 16° C. over the night, and transformed into a bio-vector bacterium S9H; firstly, positive-assumed clones were screened by an ampicillin resistant plate, and the same time, a small amount of positive-assumed clone plasmid DNA was extracted by an alkaline lysis method; then single restriction enzyme digest, double restriction enzyme digests and agarose gel electrophoresis were performed to observe and identify the size of the positive clone plasmid. The result shows that the construction of the positive recombinant plasmid pBR322-K88ac containing the fae gene operon is correct and plasmid DNA sequencing was used for verification.

The 0.8% agarose gel electrophoresis result of the PCR amplified product shows that specific target bands are amplified by PCR with a size of about 7.9 Kb, which is consistent with the size of the expected fae operon gene. The positive-assumed recombinant plasmid pBR322-K88ac was screened by an ampicillin LB plate; the enzyme digestion product of the purified recombinant plasmid DNA was subjected to agarose gel electrophoresis to indicate that the positive-assumed recombinant plasmid is the recombinant plasmid inserted with the operon containing a target gene fae; then the recombinant plasmid was verified via sequencing by Shanghai GeneCore BioTechnologies Co., Ltd. to finally construct the recombinant bio-vector bacterium S9H-K88ac containing the positive recombinant plasmid pBR322-K88ac.

### (V) Agglutination Reaction Mediated by Mouse Anti-K88ac Fimbriae Monoclonal Antibody

A single colony of the recombinant bio-vector bacterium S9H-K88ac of pBR322-K88ac was picked and inoculated on an LB medium containing 100 µg/mL ampicillin, and subjected to shaking culture over the night at 37° C. 10 µL bacterial solution was taken and respectively mixed well with the same amount of rabbit anti-K88ac fimbriae polyclonal antibody serum and mouse anti-K88ac monoclonal antibody (prepared by this laboratory) for agglutination test reaction under the light for observation. The result shows that the same as the Escherichia coli K88ac reference strain C83902, the recombinant bacterium also may cause obvious agglutination reaction with the rabbit anti-K88ac fimbriae polyclonal antibody serum and mouse anti-K88ac fimbriae monoclonal antibody after being cultured for a period of time at 37° C. over the night. The mouse anti-serum prepared by the fimbriae thermally extracted and purified from the recombinant bacterium S9H-K88ac may also cause obvious agglutination reaction with the recombinant bio-vector bacterium S9H-K88ac; the agglutinating antibody valence on the glass plate is up to 1:200. The agglutination test reaction of the negative control strain S9H-pBR322 is negative. To sum up, the results show that the bio-vector bacterium Salmonella sp. S9H expresses and carries the swine Escherichia coli antigen factor K88ac on the surface thereof.

### (VI) Observation by TEM

The recombinant vector bacterium S9H-K88ac was subjected to static culture for 24 h on an LB medium, and then centrifuged and washed with PBS solution for twice, a small amount of bacterial solution was sucked and suspended into a copper grid screen, and subjected to negative staining for 5 minutes with phosphotungstic acid, then observed and shoot under Philips Tecnai12-twin TEM. Meanwhile, the Escherichia coli K88ac reference strain C83902 and pBR322-carrying empty vector strain S9H-pBR322 respectively served as positive and negative controls.

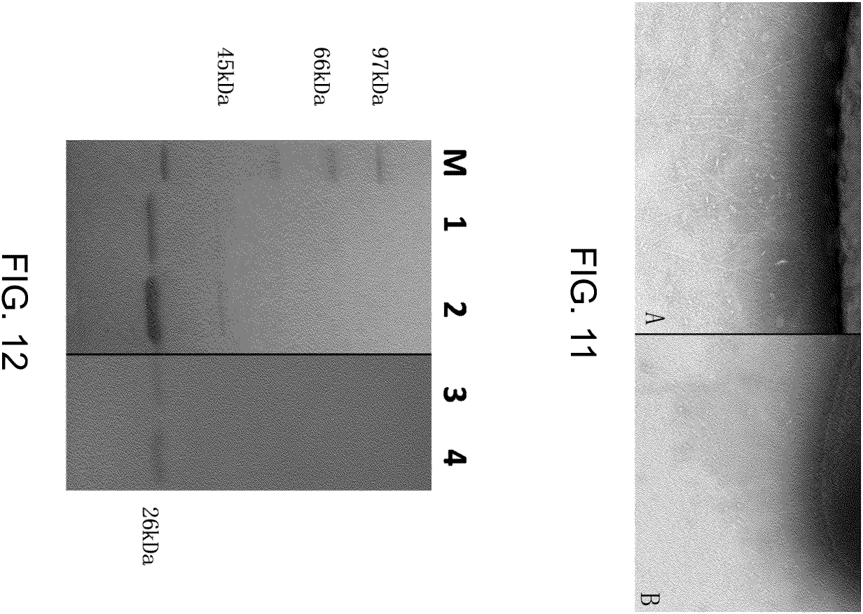

The Escherichia coli K88ac reference strain and the recombinant bio-vector bacterium S9H-K88ac were subjected to negative staining, and observed under TEM to find that lots of fimbriae (FIG. 11) are displayed on the surface of the bacteria, and the recombinant bio-vector bacteria have compact, long and thin fimbriae, indicating the better expression of fimbriae in the recombinant bacteria.

### (VII) Identification of Fimbriae

Extraction of the fimbriae from the recombinant bio-vector bacterium S9H- K88ac and the Escherichia coli K88ac reference strain: the cultured bacterial solution was centrifuged and washed with PBS twice using a heat extraction method, then suspended with a 0.05 M Tris-HCl (pH7.4)-1 M Nacl (pH7.4-7.6) low-salt solution, and treated in a water bath at 60° C. for 30 minutes, and centrifuged at 8000 rpm for 20 minutes to separate fimbriae; saturated ammonium sulfate was added to a final concentration of 25% to precipitate and purify the fimbriae, and then the obtained fimbriae was preserved for further use at 4° C.

SDS-PAGE and Western blot of the purified fimbriae from the recombinant bio-vector bacterium S9H- K88ac and the Escherichia coli K88ac reference strain: 12%SDS-PAGE was performed according to the relevant literatures to prepare into a 12% separation gel, and 5% spacer gel. The supernatant of the purified fimbriae was mixed well with 5×SDS loading buffer, boiled for 8 minutes in boiling water to denaturate the protein, the loading quantity of sample per well was 20 µL; then polyacrylamide gel electrophoresis was performed for 4 h at a constant pressure of 100 V. Coomassie Brilliant Blue R250 was used for staining to observe the size of the major structural protein bands expressing the fimbriae. The protein bands in the gel were transferred onto a NC membrane with a BIO-RAD protein strip transfer-print system for 2 h at a constant current of 300 mA. At the end of the transfer print, the NC membrane was blocked with 10% skimmed milk, staying over the night at 4° C. The NC membrane was washed with PBST for 3 times, and the washed NC membrane was put to the diluted (1:400) mouse anti-K88ac fimbriae monoclonal antibody serum for acting for 2 h at 37° C., then washed with PBST for 5 minutes for 3 times, and put to a diluted (1:50) goat-anti-mouse IgG-HRP (purchased from SHANGHAI SINO-AMERICAN BIOTECHNOLOGY CO., LTD.) for acting for 2 h at 37° C., then washed with PBST for 5 minutes for 3 times, and the obtained product was transferred into a fresh substrate DAB developing solution (10 mL PBS, 9 mg DAB, 20 µL 30%H2O2) for color developing in the dark until the band was clear, the reaction was stopped with distilled water.

The SDS-PAGE result shows that there is a major structural protein band at 26 KD from the separated and purified recombinant bacterium S9H- K88ac; and the size of the major structural protein band is consistent with the size of the major structural protein subunit of the K88ac fimbriae expressed by fae operon, and is also consistent with the size of the major structural protein band of the fimbriae heat-extracted, separated and purified from the Escherichia coli K88ac prototype strain C83902; while for the heat-extracted product of the negative control strain S9H-pBR322, there is no corresponding band at 18.5 KD after being identified by SDS-PAGE (FIG. 12: lane 1 and lane 2).Western blot result shows that the mouse anti-K88ac fimbriae monoclonal antibody may specifically identify the major structural protein bands of fimbriae expressed by the recombinant bacterium S9H- K88ac and the Escherichia coli K88ac prototype strain (FIG. 10: lane 3 and lane 4), but may not identify the heat-extracted product of the negative control strain S9H-pBR322. The above result also shows that the bio-vector bacterium Salmonella sp. S9H may express and carry the bovine Escherichia coli K88ac antigen factor on the surface thereof.

### Example 6 Test and Verification for Surface Expression and Carrying of the Human Salmonella Sp. Antigen Factor I by the Bio-Vector Bacterium Salmonella Sp. S9H

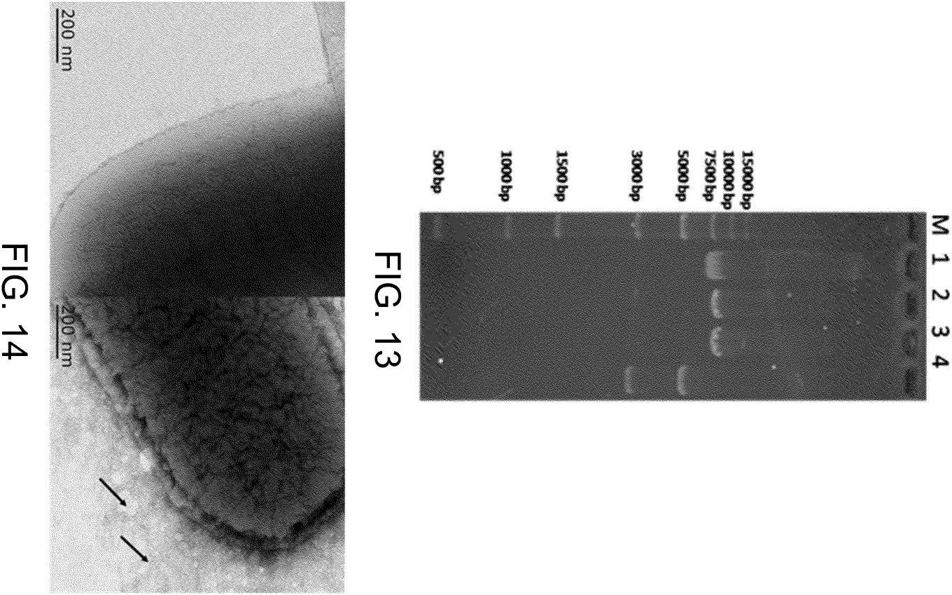

The full length operon Fim gene fragments of human Salmonella sp. of antigen factor I (type I fimbriae) were searched from the full length genome sequences published in NCBI GenBank, namely, the whole genome sequence (NCBI accession number: NZ_QRCP00000000.1) of the Salmonella enteritidis NCTR380 strain, the whole genome sequence (NCBI accession number: NZ_QRCP00000000.1) of the Salmonella enteritidis 219/11 strain, the whole genome sequence (NCBI accession number: NZ_MYTC00000000.1) of the Salmonella enteritidis BCW_4356 strain, the whole genome sequence (NCBI accession number: NZ_CP018657.1) of the Salmonella enteritidis 92-0392 strain, and the whole genome sequence (NCBI accession number: NZ_PHGY00000000.1) of the Salmonella enteritidis N152 strain to design PCR amplified primers. The restriction enzyme cutting sites of restriction enzymes BamHI and NheI, and protective bases were added at the 5′ terminals of the forward and reverse primers, respectively as follows: FimA-H UP1: 5′-AT GAA AAT TAA AAC TCT GG-3′, FimA-H LO1: 5′-TTA TTG ATA AAC AAA AGT CAC-3′. The chromosome DNA template of human Salmonella enteritidis reference strain C50336, and Long PCR high-fidelity DNA polymerase from Roch were taken, and the PCR amplified product was recovered and purified by an agarose gel recovery kit. The pBR322 expression plasmid was extracted by a plasmid extraction kit, and the pBR322 plasmid and the operon fim gene amplified product were respectively subjected to agarose gel electrophoresis for observation and identification; the recovery product of the agarose gel was subjected to BamHI and NheI double restriction enzymes digests, and extracted by phenol/chloroform, precipitated with ethanol and purified; the PCR amplified product after through double restriction enzymes digests was mixed with the pBR322 plasmid (pBR322-I) according to the ratio of 3:1 at the same time; the mixture was ligated by a T4 DNA ligase at 16° C. over the night, and electro-transformed into S9H competent cells of the bio-vector bacterium Salmonella sp. S9H. Specific operation was as follows: 2 µL I-pBR322 plasmid mixture was taken and mixed with 40 µL S9H electro-transformed competent cells, then put on an ice bath for 30 minutes at 4° C. ; the above mixture was added to a Bio-Rad electric shock for electro-transformation; then the transformed products were rapidly transferred onto a 1 mL SOC medium, shaken for 4 h at 37° C. and centrifuged for 10 minutes at 4000 rpm to discard supernatant; and a few of bottom liquid was reserved and resuspended, and then cultured on an ampicillin plate medium at 37° C. to screen the bacterial colonies of the positive-assumed recombinant bio-vector bacterium Salmonella sp. S9H-I; the recombinant plasmid was extracted and subjected to BamHI and NheI single restriction enzyme digest and double restriction enzymes digests, then subjected to agarose gel electrophoresis for observation and identification (FIG. 13); the recombinant plasmid pBR322-I DNA sequencing was performed for verification, and the recombinant bio-vector bacterium Salmonella sp. S9H-I was preserved.

A single bacterial colony of the recombinant bio-vector bacterium S9H-I of pBR322-I was picked and inoculated onto an LB medium containing 100 µg/mL ampicillin for shaking culture over the night at 37° C.; 10 µL bacterial solution was taken and respectively mixed with the same amount of polyclonal antibody serum (prepared by this laboratory) of the mouse anti-I antigen factor (type-I fimbriae) for agglutination test reaction under the light for observation. The result shows that the same as the Salmonella enteritidis reference strain C50336, the recombinant bacterium may cause obvious agglutination reaction with the polyclonal antibody serum of the mouse anti-I antigen factor (type-I fimbriae). The agglutination test reaction of the negative control strain S9H is negative. The above agglutination test reaction shows that the bacterium S9H-I expresses and carries the human Salmonella sp. antigen factor I on the surface thereof.

A single colony of the recombinant bio-vector bacterium S9H-I of pBR322-I was picked and inoculated onto an LB medium containing 100 µg/mL ampicillin for shaking culture over the night at 37° C. The single colony was picked and respectively inoculated onto LB and ampicillin resistant LB liquid media, and placed at 37° C. for shaking culture for 12 h, and subjected to blind passage for 2 generations; a small amount of bacterial solution was sucked to be respectively inoculated onto LB and ampicillin resistant LB liquid media for static culture for 48 h, then centrifuged for 2 minutes at 10000 rpm; precipitates were resuspended with sterile PBS; and a small amount of bacterial solution was sucked, and subjected to negative staining and observed under TEM. Netherlands Philips Tecnai 12 TEM was used for observation, shooting and result display. The result shows that the recombinant bio-vector bacterium S9H-I carries an I antigenic component (type-I fimbriae) on the surface thereof, while an I antigen factor component (type-I fimbriae) (FIG. 14) seemed to be not found on the surface of the negative control bacterium S9H.

1. A generic inert bio-vector Salmonella sp., wherein the generic inert bio-vector Salmonella sp. is derived from a continuous in-vitro culture of an inert bio-vector bacterium Salmonella sp. S9 by using LB solid and liquid culture media for passage to the fortieth generation and above, the strain derived from passage 40th to 60th generation is named as a generic inert bio-vector S9H, and the deposit number of the inert bio-vector bacterium S9 is CGMCC No. 17340.

2. A method for obtaining the generic inert bio-vector Salmonella sp. according to claim 1, wherein the method comprises the following steps: the generic inert bio-vector Salmonella sp. is a strain derived from a continuous in-vitro culture of an inert bio-vector bacteria Salmonella sp. S9 by using LB solid and liquid culture media for passage to the 40th and 60th generation.

3. A generic inert bio-vector indirect agglutination test detection system, wherein the detection system comprises the generic inert bio-vector Salmonella sp. according to claim 1 and a complex that may display, express and carry a specific antigen factor on the surface thereof.

4. The detection system according to claim 3, wherein the specific antigen factor is one or more from a group consisting of a P factor of poultry Salmonella sp., a K88ac antigen factor of porcine Escherichia coli, a K99 antigen factor of bovine Escherichia coli and an I antigen factor of human Salmonella sp..

5. A method for construction of the generic inert bio-vector indirect agglutination test detection system according to claim 3, comprising the following steps:
1) obtaining a coding gene of a specific antigen factor;
2) ligation of the coding gene of the specific antigen factor with an expressing plasmid to obtain a recombinant plasmid;
3) transformation of the recombinant plasmid into an S9H electrocompetent cell to obtain an identified recombinant strain as the generic inert bio-vector indirect agglutination test detection system.

6. The method for construction of the generic inert bio-vector indirect agglutination test detection system according to claim 5, wherein the coding gene of the specific antigen factor in the step 1) is the coding gene for a P factor of poultry Salmonella sp., the coding gene for a K88ac antigen factor of porcine Escherichia coli, the coding gene for a K99 antigen factor of bovine Escherichia coli or the coding gene for an I antigen factor of human Salmonella sp..

7. (canceled)

8. (canceled)

9. (canceled)

10. A detection kit, wherein the detection kit comprises the generic inert bio-vector Salmonella sp. according to claim 1 .

11. A method for construction of the generic inert bio-vector indirect agglutination test detection system according to claim 4, comprising the following steps:
1) obtaining a coding gene of a specific antigen factor;
2) ligation of the coding gene of the specific antigen factor with an expressing plasmid to obtain a recombinant plasmid;
3) transformation of the recombinant plasmid into an S9H electrocompetent cell to obtain an identified recombinant strain as the generic inert bio-vector indirect agglutination test detection system.

12. A detection kit, wherein the detection kit comprises the detection system according to claim 3.
